(19) United States
(12) Patent Application Publication
(10) Pub. No.: US 2026/0325209 A1
(43) Pub. Date: Oct. 22, 2026

(54) DEVICES AND METHODS FOR PRIMING SOLID TUMORS WITH PRESSURE PULSES TO ENHANCE ANTICANCER THERAPIES

(71) Applicants: LASERLEAP TECHNOLOGIES, S.A., Coimbra (PT); UNIVERSIDADE DE COIMBRA, COIMBRA (PT)

(72) Inventors: Luís Guilherme DA SILVA ARNAUT MOREIRA, COIMBRA (PT); Maria Inês PIMENTEL MENDES, COIMBRA (PT); Diogo António DA CRUZ FIGUEIREDO PEREIRA, COIMBRA (PT); Carlos Alberto LOURENÇO DE SERPA SOARES, Coimbra (PT); Celso Manuel DE FIGUEIREDO PAIVA JOÃO, Coimbra (PT); João Pedro FREIRE BARRACA CARDOSO SANTOS, Coimbra (PT)

(21) Appl. No.: 19/105,962

(22) PCT Filed: Aug. 21, 2023
(86) PCT No.: PCT/IB2023/058316

§ 371 Date: Feb. 24, 2025

Related U.S. Application Data
(63) Continuation of application No. 17/892,355, filed on Aug. 22, 2022.

(30) Foreign Application Priority Data
Jul. 31, 2023 (PT) 118846

(51) Int. Cl. A61B 18/26; A61B 17/00; A61B 18/00; A61K 39/00; A61K 39/395; A61K 41/00; A61N 7/00; A61P 35/00

(52) U.S. Cl. CPC A61B 18/26; A61K 39/3955; A61K 41/0033; A61N 7/00; A61P 35/00; A61B 2017/00185; A61B 2017/0019; A61B 2018/00982; A61B 2018/266; A61K 2039/507; A61N 2007/0004

(57) ABSTRACT

The present disclosure is directed to devices for, and method of, priming a tumor microenvironment with pressure pulses to enhance the efficacy of anticancer therapeutic agents, in a subject in need thereof. Further, increased response of solid tumors locally exposed to stress waves to systemically administered therapeutic agents, is disclosed. A device according to the present disclosure comprises: a pulsed laser system (1), a light guide (2) to direct laser pulses to one or more light-to-pressure transducers (3), the one or more light-to-pressure transducers absorbing laser pulses from the pulsed laser system and generating pressure pulses, a tumor-positioning support structure (4) configured to couple one or more light-to-pressure transducers with a solid tumor (5), and a control system (6) to limit the exposure of the solid tumor to the pressure pulses. Anticancer therapeutic agents may be administered before, after or during the priming of solid tumors with pressure pulses.

### FIELD OF THE INVENTION

The present disclosure relates generally to the field of remodeling (or priming) of a tumor microenvironment (TME) to enhance the efficacy of anticancer therapeutic agents. More specifically, the disclosure relates to a pressure-pulse tumor-priming device and method of using same, and methods of priming a tumor microenvironment with pressure pulses, thereby enhancing the efficacy of anticancer therapeutic agents.

### BACKGROUND OF THE INVENTION

Active stimulation of the host immune system to mount antitumor immune responses capable of controlling tumor growth and dissemination has been pursued for a century and recently reached the status of first-line treatment for cancer indications [1-3]. A notable example is immune checkpoint blockade (ICB) therapy, which employs monoclonal antibodies (mAb) to block inhibitory immunoreceptors (checkpoints) and unlock the antitumor function of immune cells present in the immunosuppressive tumor microenvironment (TME). To promote a therapeutic response, CD8⁺T cells that are strongly activated by tumor antigens must be unrestrained by negative regulators. These negative regulators have been called “checkpoints” since they detect, resist and reverse overreaction. While checkpoint inhibitors are essential to maintain self-tolerance and prevent autoimmune diseases, they can be hijacked by tumor cells to escape immunosurveillance. Inhibitory immunoreceptors include, but are not limited to, CTLA4 (cytotoxic T-lymphocyte-associated protein 4), PD1 (programmed cell death protein 1), LAG3 (lymphocyte-activation gene-3), TIM3 (T-cell immunoglobulin and mucin domain-3), TIGIT (T-cell immunoreceptor with Ig and ITIM domains), ICOS (inducible T-cell co-stimulatory receptor), BTLA (B and T lymphocyte attenuator) and VISTA (V-domain Ig-containing Suppressor of T cell Activation). CTLA4 and PD1 are the most potent examples of T cell immune checkpoint molecules known today.

The sequence of FDA clinical approvals of ICBs is very impressive [4]: ipilimumab (2011), pembrolizumab (2014), nivolumab (2014), atezolizumab (2016), durvalumab (2017), avelumab (2017), cemiplimab (2018), dostarlimab (2021), tislelizumab (2021), relatlimab (2022). In China other ICBs have been approved for clinical use, such as: toripalimab (2018), camrelizumab (2019), sintilimab (2019). Moreover, some of these ICBs have been granted approval for the treatment of different types of cancer. This remarkable progress in immunotherapies explains why in 2019 it was estimated that the percentage of US cancer patients who were eligible for treatment with ICBs was ~44%. However, the percentage of patients with cancer that do not respond to ICBs is more than 87% [5]. There is a dramatic need to improve the efficacy of immunotherapies.

CTLA4 molecules are contained within intracellular vesicles in naïve T cells and constitutively expressed on the surface of CD4⁺CD25⁺regulatory T (T_{reg}) cells. Naïve T cells are activated when their T cell receptors bind to their cognate antigen presented by antigen-presenting cells (APCs) in the presence of a co-stimulatory signal. This co-stimulatory signal is the binding between CD28 expressed on the surface of the T cell with B7 molecules (B7.1, also named CD80, or B7.2, also named CD86) on APCs. APCs are immune cells that process and present antigens for recognition by T cells, and include B lymphocytes, dendritic cells, macrophages and other immune cells. CD28 and CTLA4 compete for binding to B7-1 and B7-2 on APCs but CTLA4 binds to B7-1 and B7-2 more tightly and delivers negative rather than costimulatory signals to the T cells. CTLA4 counteracts several internal signaling nodes to impede activation and proliferation of T cells [6]. T_{reg} cells, which constitutively express CTLA4, can arrest T cell responses. The recognition that CTLA4 is a negative regulator of T cell activity suggested that blocking its actions could rescue T cells response to cancer cells. Indeed, neutralizing anti-CTLA4 mAbs enhance antitumoral immunity. In addition to boosting CD8⁺effector T cell responses, anti-CTLA4 therapy depletes local intra-tumoral T_{reg} cells through antibody-dependent cell-mediated cytotoxicity and shifts the balance of the TME away from immunosuppression [7].

Human PD1 is expressed on T cells after T cell receptor stimulation, and binds to the B7 homologues PDL1 and PDL2, which are constitutively expressed on APCs and can be induced in non-hematopoietic tissues. PDL1 (programmed cell death ligand 1, also known as B7-H1), is a transmembrane protein that down-regulates immune responses through binding to its two inhibitory receptors PD1 and B7.1, and is present on many cell types, including T cells, tumor cells, epithelial cells and endothelial cells, much more frequently than PDL2 (programmed cell death ligand 2, also known as B7-DC). PD1 restrains immune responses primarily through inhibitory signaling in CD8⁺effector T cells and in T_{reg} cells [7]. When PD1 engages its ligands, it can induce a state of T cell dysfunction called T cell exhaustion. Tumor cells can upregulate PD1 ligands. PDL1, expressed by cells in the TME, engages PD1 on T cells and subsequently triggers inhibitory signaling, blocking effector functions and reducing T-cell killing capacity. Hence, tumor cells can induce T cell exhaustion and generate a TME that facilitates tumor growth and invasion. As the PD1 /PDL1 pathway protects cells from T cell attack, anti-PD1 and anti-PDL1 antibodies can enhance the functional properties of CD8⁺effector T cells at the tumor site.

Activation of T cells allows T cell lymphocytes to recognize an antigen on a specific target cell. Activated CD8⁺T cells gradually transform into effector T cells (or cytotoxic T lymphocytes, CTLs), which recognize target cells and kill them by distinct pathways. In one pathway, the Fas ligand (or CD95L) expressed on the surface of CTL binds to the Fas receptor (or CD95) on the target cell and triggers apoptosis through the caspase cascade. In another pathway, the CTL releases granulysin, perforins, cathepsin C and/or granzymes into the intercellular space between the CTL and the target cell, which are highly cytotoxic to the target cell. Although the mechanisms of these immunotherapies are complex, interrelated and still under investigation, these pathways show that the anti-tumor activity of CTLs requires their infiltration in solid tumors. The same requirement of TME infiltration also applies to other immunotherapies, such as adoptive T cell therapy or cancer vaccines.

The failure of immunotherapies in the treatment of the vast majority of patients, and the remarkable success in a minority of them, has been subject to intense scrutiny. Absences of therapeutic benefits have been attributed to a variety of factors, including the abnormal TME, characterized by dysfunctional blood vessels that hinder the delivery of immunotherapeutic agents and cause immunosuppression. Indeed, a spatiotemporal lack of sufficient tumor blood perfusion can result in hypoxia, low pH, and inadequate delivery of medicines, which in turn compromises the efficacy of cancer therapies, including immunotherapy. The difficulty of anticancer drugs to access all of the cells in the TME is recognized in chemotherapy [8] and can be expected to be a more limiting factor for biologics and immunotherapies in view of the sizes of the corresponding therapeutic agents. The rapid proliferation of cancer cells forces blood vessels apart in solid tumors, reduces vascular density, compresses blood and lymphatic vessels, limits the delivery of oxygen and nutrients, builds-up products of metabolism and reduces pH. The difficulty to infiltrate solid tumors is aggravated when mAbs are employed because they are very large macromolecules, or associations of macromolecules, with molecular weights ~150 kDa. The physicochemical properties and large size of mAbs hinder their passive diffusion through vascular epithelial cells. The main mechanism by which mAbs distribute from the body into the tissue is by convective transport, i.e., by the blood-tissue hydrostatic gradient. The affinity of the mAbs to target antigens within the interstitial space or on cell surface and their convection into the lymph, determine mAbs retention in tissues. The efficiency of convection into the interstitial space is much lower than the efficiency of convection out of it. A consequence is that the clinical volume of the central compartment of most mAbs is in the range of 2-3 L, similar to vascular water, and the overall volume of distribution in the steady state is 8-20 L [9]. The limited volumes of distribution of mAbs, and in particular of ICBs, show that they are largely confined to the vascular compartment [10].

The increased leakage from the blood vessels in solid tumors and the inability of the intratumor lymphatics to drain this fluid effectively has been explored by nanoparticle delivery systems to accumulate therapeutic agents in the tumor using the enhanced permeation retention effect [11]. Nanoparticulate drug carriers have been shown to mediate greater tumor drug deposition compared with free drugs, and the delivered drug can persist for days at concentrations that exceed peak tumor concentrations achieved with free drug. However, nanomedicines tend to accumulate in tissues of the reticuloendothelial system (spleen, liver, lungs) and tend to stay in the vicinity of the tumor vascular fenestrae [12]. There is a low probability that nanomedicines reach a majority of target cells within the tumor.

The mechanical microenvironment of solid tumors is characterized by elevated interstitial fluid pressure (IFP) and by solid stress. These two contributions to stress in the TME are distinguished by the fact that IFP is the isotropic stress exerted by fluids from increased leakage and poor drainage in the tumor, whereas solid stress is exerted by nonfluid components. IFP is originated by the high vascular permeability of the TME coupled with mechanical compression of downstream blood vessels and draining lymphatic vessels. The solid stress is associated with the hyperproliferation of cancer cells, which exert a force against nearby structural elements of tumor and normal tissue that according to the law of action-reaction, exert a force with equal magnitude but opposite direction. Vessel compression has two consequences for therapeutics: (i) the collapse of blood vessels hinders the access of drugs and immune cells, (ii) the lack of lymphatic vessel function reduces drainage, increases IFP and the transport of large therapeutics (e.g., antibodies that block inhibitory checkpoint molecules) or nanomedicines becomes diffusional and is reduced because of their large size. These consequences are aggravated in immunotherapies that depend on the infiltration of tumor antigen-specific cytotoxic T lymphocytes into solid tumors, including in adoptive T cell therapies and in cancer vaccines.

The abnormal and disorganized tumors vasculature has been targeted by various therapies, namely by antiangiogenic agents such as anti-vascular endothelial growth factor (anti-VEGF). At low doses, antiangiogenic treatment normalizes tumor vasculature, improves tumor perfusion and drug delivery [13]. Therapies to overcome IFP and high growth-induced solid stress of solid tumors are less common. IFP is in the range 0-3 mmHg in normal tissue. However, fluid leaking from the blood vessels in solid tumors and the inability of the intratumor lymphatics to drain this fluid effectively, leads IFP to reach values in the range 5-40 mmHg, and even 75-130 mmHg in dermoplastic pancreatic tumors [14], although it drops abruptly at the tumor margins. Anti-solid stress strategy is distinct from the vessel normalization strategy, which employs anti-angiogenic agents to prune immature vessels and fortify the remaining vessels. Anti-solid stress aims at decompressing vessels to increase perfusion [15]. If it succeeds also in lowering the venous resistance and re-establishing lymphatic drainage, it will also reduce IFP. IFP and solid stress contribute to limit anticancer drugs to reach tumors cells inside a solid tumor. The limits imposed to the delivery of immune cells to solid tumors are even tighter. This can have dramatic effects in the efficacy of immunotherapies. In particular, CD8⁺T cells can be excluded from, or trapped within, tumors by the dense, fibrotic extracellular matrix produced by cancer-associated fibroblasts [16]. The infiltration of CTLs in tumors correlates with the therapeutic efficacy of ICBs.

In a pharmacological approach to reduce solid stress, tumor-targeted angiotensin receptor blockers were shown to reduce the activity of cancer-associated fibroblasts, and when combined with ICBs displayed enhanced efficacy [17]. For example, the combination of angiotensin receptor blockers with an ICB mixture of aCTLA-4 plus aPD-1 increased the median survival of mice with orthotopic 4T1 tumors from 17 days in controls to 24 days in the combination (the median survival of mice treated with aCTLA-4 plus aPD-1 was 20 days) [17]. Although such pharmacological approaches can reduce solid stress in solid tumors and increase the efficacy of immunotherapies, they have systemic effects.

The effects of elevated IFP and of solid stress may be considered less important in chemotherapies employing small molecule drugs, because of their higher diffusion coefficients. However, this consideration ignores the fact that most of the small molecule drugs are weakly soluble or insoluble in aqueous media, and are extensively bound to plasma proteins, such as albumin and low-and high-density lipoproteins, shortly after administration. For example, human serum albumin (HSA) has a molecular weight ~67 kDa, which is nearly half the molecular weight of a mAb, and small molecule drugs bound to HAS may have the same problems to infiltrate solid tumors as macromolecules, nanomedicines, biologicals or CTLs.

Considering that the consequences of vascular normalization and solid-stress alleviation are interrelated, and that both contribute to enhanced drug delivery into solid tumors, the term “tumor priming” was suggested to designate both these strategies [14]. Drug penetration into solid tumors was also enhanced using photodynamic tumor priming, where a photosensitizer is employed in sub-tumoricidal concentrations to enhance tumor permeability to chemo and biological agents [18]. This offers spaciotemporal control of solid tumor permeability with low-toxicity photosensitizers, but remains a pharmacological approach that requires the use of an additional medicine. Acoustic priming of solid tumors using focused ultrasound has also been described [19]. The application of focused ultrasound to biological tissues is associated with the generation of thermal and cavitation effects that cause changes in target cell physiology [19].

Acoustic priming therapy uses piezoelectric transducers configured to produce spatial-peak temporal-average intensities (ISPTA) between 10 and 1000 W/cm² in the treatment zone and ultrasound with frequencies between 0.01 and 10 MHz, and the ultrasound is applied continuously from a time in the range of 0.5 to 5 seconds for any particular volume of the treatment zone [19]. The values of ISPTA in acoustic priming therapy largely exceed the current FDA output limits for diagnostic ultrasound: ISPTA<0.72 W/cm².

Current pharmacological approaches to solid tumor priming met with some success but are not without off-target effects. Radiological approaches to solid tumor priming increase the exposure of the body to harmful radiation. Acoustic tumor priming requires ultrasound waves with spatial-peak temporal-average intensities that largely exceed the output safety limits set by FDA for diagnostic ultrasound. This disclosure relates to, for the first time, the use of pressure pulses with spatial-peak temporal-average intensities within the output safety limits set by the FDA for diagnostic ultrasound, to achieve solid tumor priming and increase tumor response to therapeutic agents.

### SUMMARY OF THE INVENTION

The use of pressure pulses in tumor-priming therapy (PPTPT) is disclosed herein. In one embodiment, PPTPT uses laser light pulses and piezophotonic (light-to-pressure) transducers to generate high-pressure and broadband photoacoustic waves that traverse a solid tumor and enhance the infiltration of chemo/biological/immunological agents. In another embodiment, PPTPT uses laser light pulses to ablate the surface of a material and generate shock waves that pass through a solid tumor and facilitate the infiltration of chemo, biological, immunological agents, or any combination thereof, in the tumor. PPTPT combines the exposure of the solid tumor to pressure pulses with the administration of chemotherapy, biologicals or immunotherapies. The pressure pulses work mainly by priming the solid tumor so that the chemo/biological/immunological agents infiltrate deeply into the solid tumor and, consequently, the response of the tumor to the chemo, biological, immunological agents, or any combination thereof, is stronger.

In one aspect of the present disclosure, there is provided a device for tumor priming by pressure pulses comprising: a pulsed laser system with a pulse repetition rate from 0.1 Hz to 100 Hz; a light guide configured to direct laser pulses to one or more light-to-pressure transducers; one or more light-to-pressure transducers configured to absorb laser pulses from the pulsed laser system and generate pressure pulses, where the pressure pulses have peak compressional pressures from 0.1 MPa to 100 MPa, and 90% of each pressure pulse lasts from 0.1 ns to 500 ns; a tumor-positioning support structure configured to couple one or more light-to-pressure transducers with a selected area from a solid tumor, at a distance shorter than 3 cm from the area; and a control system configured to limit a exposure of the solid tumor to the pressure pulses for a period of time from 1 second to 60 minutes.

In a further embodiment, the light guide comprises one or more optical fibers or light pipes.

In a further embodiment, the light guide comprises mirrors, lenses, prisms, diffusers, polarizers, or any combination thereof.

In a further embodiment, the light-to-pressure transducer comprises a laser light absorbing system and a material with a Grüneisen parameter higher than 0.5, and wherein each pressure pulse is a wavefront of a photoacoustic wave.

In a further embodiment, the light-to-pressure transducer comprises a laser light absorbing system and a material with an ablation threshold below 200 mJ/cm², and wherein each pressure pulse is a wavefront of a shock wave.

In a further embodiment, the tumor-positioning support structure is configured to hold one or more light-to-pressure transducers together with an acoustic coupling element disposed between the transducers and the surface of a solid tumor.

In a further embodiment, the tumor-positioning support structure is an endoscope and the light guide is one or more optical fibers configured to carry a laser light from a light source through the endoscope, to one or more light-to-pressure transducers at the distal ends of the optical fibers.

In a further embodiment, the endoscope is configured for insertion in a hollow organ through a natural body opening or through an incision in the body with less 2 cm in length.

In a further embodiment, the tumor-positioning support structure is a catheter and the light guide is one or more optical fibers configured to carry a laser light from a light source through the catheter, to one or more light-to-pressure transducers at the distal ends of the optical fibers.

In a further embodiment, the catheter is configured for insertion into a body cavity, duct, vessel, brain, skin or adipose tissue.

In a further embodiment, the tumor-positioning support structure comprises a sharp end configured to enable the insertion of one or more light-to-pressure transducers into the solid tumor.

In another aspect of the disclosure, there is provided a method for treating a solid tumor in a subject afflicted with cancer, the method comprising: pressure-pulse tumor priming of the solid tumor by exposure of the solid tumor to one or more pressure pulses, wherein the pressure pulses have peak compressional pressures from 0.1 MPa to 100 MPa, and 90% of each pressure pulse lasts from 1 ns to 500 ns; and administering of one or more anticancer therapeutic agents to the subject, thereby treating the solid tumor in a subject afflicted with cancer. In a further embodiment, the pressure-pulse tumor priming of the solid tumor is performed with the device according to the present disclosure.

In a further embodiment, the method further comprises a step of repeating the pressure-pulse tumor priming, the administering of one or more anticancer therapeutic agents, or both, at least one time with doses that improve a response of the solid tumor to the treatment.

In a further embodiment, the anticancer therapeutic agent is selected from the group consisting of an inhibitor to an inhibitory checkpoint molecule, an activator of a stimulatory checkpoint molecule, an antibody, a cytokine, an interferon, an interleukin, a vaccine, an oncolytic virus, chimeric antigen receptor T cells, and any combination thereof.

In a further embodiment, the therapeutic agent is a biological therapeutic.

In a further embodiment, the therapeutic agent is a monoclonal antibody (mAb) used to treat cancer, or any combination of mAbs used to treat cancer.

In a further embodiment, the therapeutic agent is selected from ipilimumab, pembrolizumab, nivolumab, atezolizumab, durvalumab, avelumab, cemiplimab, dostarlimab, tislelizumab, relatlimab, toripalimab, camrelizumab, sintilimab, or any combination thereof.

In a further embodiment, the therapeutic agent is a cytostatic or a cytotoxic drug bound to a plasma protein.

In a further embodiment, the therapeutic agent is a macromolecule.

In a further embodiment, the therapeutic agent is a nanomedicine.

In another aspect of the disclosure, there is provided a device comprising means for generating pressure pulses having peak compressional pressures from 0.1 MPa to 100 MPa, and 90% of each pressure pulse lasts from 1 ns to 500 ns, for treating a solid tumor in a subject afflicted with cancer.

In a further embodiment, the device is the device according to the present disclosure.

In another aspect of the disclosure, there is provided a system comprising at least one device according to the present disclosure.

In another aspect of the disclosure, there is provided a kit comprising a device according to the present disclosure, and an anticancer therapeutic agent.

In a further embodiment, the anticancer therapeutic agent is selected from the group consisting of an inhibitor to an inhibitory checkpoint molecule, an activator of a stimulatory checkpoint molecule, an antibody, a cytokine, an interferon, an interleukin, a vaccine, an oncolytic virus, chimeric antigen receptor T cells, and any combination thereof.

In a further embodiment, the therapeutic agent is a biological therapeutic.

In a further embodiment, the therapeutic agent is a monoclonal antibody (mAb) used to treat cancer, or any combination of mAbs used to treat cancer.

In a further embodiment, the therapeutic agent is selected from ipilimumab, pembrolizumab, nivolumab, atezolizumab, durvalumab, avelumab, cemiplimab, dostarlimab, tislelizumab, relatlimab, toripalimab, camrelizumab, sintilimab, or any combination thereof.

In a further embodiment, the therapeutic agent is a cytostatic or a cytotoxic drug bound to a plasma protein.

In a further embodiment, the therapeutic agent is a macromolecule.

In a further embodiment, the therapeutic agent is a nanomedicine.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present disclosure will become apparent from the detailed description given hereinafter. However, it should be understood that the detailed description and specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced. The dimension of the components and features shown in the figures are chosen for convenience and clarity and are not necessarily shown to scale.

FIGS. 1A-B present absolute pressure pulses generated by piezophotonic materials made of carbon nanoparticles and PDMS when excited with laser fluences of (FIG. 1A) ~60 mJ/cm² and (FIG. 1B) ~126 mJ/cm² and 8 ns duration, detected with a hydrophone calibrated to the 1 to 30 MHz range;

FIG. 2 presents Fourier transform of a stress wave generated by a piezophotonic material made of carbon nanoparticles and polymer when excited with a laser fluences ~60 mJ/cm² and 8 ns duration, measured with a 225 MHz contact transducer;

FIG. 3 presents in vitro viability of immortalized monkey fibroblast (COS-7) cells in control (CTR) cell culture plates and in plates exposed to photoacoustic waves for 5 minutes (5 mins) or for 10 minutes (10 mins), at the laser repetition rates of 6 Hz or 20 Hz. The viability of the cells is not compromised by exposure of up to 12000 pressure pulses;

FIG. 4 presents magnetic resonance imaging (MRI) of tissues in the region of the neck of a Sprague Dawley rat. The left side was exposed for 5 minutes, 5 times a week for 4 weeks to photoacoustic waves with peak compressional pressures of ~3 MPa at a pulse repetition rate of 20 Hz. No differences were observed in the left side relative to the right side, which was not exposed to photoacoustic waves. No adverse effects were observed in the area where photoacoustic waves were applied;

FIG. 5 presents hematoxylin and eosin stain of tissues in the region of the left carotid of a Sprague Dawley rat exposed for 5 minutes, 5 times a week for 4 weeks, to photoacoustic waves with peak compressional pressures of ~3 MPa at a pulse repetition rate of 20 Hz. No anomalies were detected in carotids and surrounding tissues. No adverse effects were observed in the area where photoacoustic waves were applied;

FIG. 6 is a schematic cross-sectional view, not to the scale, of a tumor-priming device, according to some embodiments of the present disclosure, comprising a pulsed laser system (1) with a control system (6) to limit the number of laser pulses generated, a light guide (2) to direct the laser pulses to a light-to-pressure transducer (3). The tumor-positioning support structure (4) places the light-to-pressure transducer (3) in the proximity of a selected area of a solid tumor (5) growing in the middle of healthy tissue (7). Each pressure pulse generated by the absorption of one laser pulse by the light-to-pressure transducer, crosses a small (less than 3 cm) path of tissue or acoustic coupling medium and then traverses at least part of the tumor mass;

FIG. 7 is a schematic cross-sectional view, not to the scale, of a tumor-priming device, according to some embodiments of the present disclosure, to generate pressure pulses in the vicinity of a solid tumor using endoscopy, comprising a pulsed laser system (1) with a control system (6) to limit the number of laser pulses generated, a light guide (2) to direct the laser pulses to a light-to-pressure transducer (3). The tumor-positioning support structure (4) is an endoscope that places the light-to-pressure transducer (3) at less than 3 cm of a selected area of a solid tumor (5) growing in the middle of healthy tissues in the abdomen. Each pressure pulse is directed to the light-to-pressure transducer where it generates a pressure pulse that crosses the intestinal wall and reaches the tumor;

FIG. 8 is a schematic cross-sectional view, not to the scale, of a tumor-priming device, according to some embodiments of the present disclosure, to generate pressure pulses in the vicinity of a solid tumor using a catheter, comprising a pulsed laser system (1) with a control system (6) to limit the number of laser pulses generated, a light guide (2) possibly with a lens at the distal end to direct the laser pulses to a light-to-pressure transducer (3). The tumor-positioning support structure (4) is a urinary catheter with a balloon (8) that places the light-to-pressure transducer (3) at less than 3 cm of a selected area of a solid tumor (5) growing in the bladder. Each pressure pulse is directed to the light-to-pressure transducer where it generates a pressure pulse that crosses the bladder and reaches the tumor; and

FIG. 9 is Kaplan-Meier plot of BALB/c mice with orthotopic 4T1 tumors. Control group (dashed-dotted line, no priming and no therapy), group with tumors exposed to photoacoustic waves (dashed line, priming but no therapy), group treated with intraperitoneal administration of aCTLA4 (full line, no priming but therapy) and group with tumors exposed to photoacoustic waves and treated with intraperitoneal administration of aCTLA 4 (dotted line, priming and therapy). Day 0 is the day when the procedures started, chosen that the longest diameter of the tumor was at least 3 mm. The mice were sacrificed when the longest diameter of the tumor reached 12 mm.

### DETAILED DESCRIPTION OF THE INVENTION

For the purpose of this disclosure, the following definitions will apply:

The term “therapeutic agent” refers to a small molecule drug or a biologic drug or an immune cell, that can be used to treat a tumor, and include a chemotherapeutic drug, a radiosensitizer, a photosensitizer, a nanoparticle, a senolytic agent, a biological, an immunomodulatory agent, an immune molecule, or CD8⁺T cells activated by tumor antigens and unrestrained by negative regulators. The therapeutic agent can be an agent approved by a regulatory agency for treating tumors or cancer, undergoing clinical trials prior to regulatory approval, or that is under investigation for treating tumors or cancer.

The term “small molecule drug” refers to an organic compound having a molecular weight equal or less than 1 kDa. The term includes drugs having desired pharmacological properties and includes compounds that can be administered orally or by injection. Small molecule drugs include cytostatic or cytotoxic drugs used in chemotherapy of cancer.

The term “photosensitizer” refers to a dye, possibly bound to a targeting moiety, that has no detectable therapeutic effect in the electronic ground state but when electronically excited can trigger processes that eventually lead to cell death, as illustrated by the photogeneration of reactive oxygen species in photodynamic therapy of cancer and by photoimmunotherapy.

The term “macromolecule” refers to an organic, or bioorganic, molecule with a molecular weight higher than 1 kDa, which can be a protein, a bioconjugate, a RNA molecule or a DNA molecule, or a fragment of the molecules.

The terms “biologicals” or “biological therapeutic” refer to a diverse group of medicines which includes vaccines, growth factors, immune modulators, monoclonal antibodies, as well as products derived from human blood and plasma. This definition specifically includes proteins purified from living culture systems or from blood.

The term “immunomodulatory agent” refers to a checkpoint inhibitor, a co-stimulator of immune pathways, an antibody targeting immune cell antigens and/or cancer antigens, and cell therapy approaches (e.g., adoptive cell transfers with genetically modified receptors such as chimeric antigen receptor therapies), and specifically includes immunomodulatory agents such as ipilimumab, pembrolizumab, nivolumab, atezolizumab, durvalumab, avelumab, cemiplimab, dostarlimab, tislelizumab, relatlimab, toripalimab, camrelizumab and sintilimab.

The term “nanomedicine” refers to a nanoparticulate drug carrier system with a size in the range of 1 nm to 500 nm in one or more external dimensions, for more than 50% of the particles, according to the number size distribution, incorporating a small molecule drug, a photosensitizer or a macromolecule.

The term “tumor priming” refers to vascular normalization and solid-state alleviation in solid tumors, including subsequent or concomitant reduction of interstitial fluid pressure inside the solid tumor, to facilitate the infiltration of therapeutic agents into a solid tumor.

The terms “piezophotonic transducer” or “light-to-pressure transducer” refer to a material that substantially absorbs the light of a laser pulse and transforms the optical energy absorbed into a pressure pulse.

The term “pressure pulse” refers to a perturbation that carries temporary density changes inside the medium where it propagates. This definition of “pressure pulse” covers explicitly both shock waves and photoacoustic waves, which are also designated collectively as “stress waves”.

The term “pressure-pulse tumor priming” refers to tumor priming by stress waves, which can be photoacoustic and/or shock waves.

The terms “comprises”, “comprising”, “includes”, “including”, “having” and their conjugates mean “including but not limited to”.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

The term “subject” as used herein refers to an animal, more particularly to non-human mammals, human, and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In one embodiment, the subject is a human.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

According to some embodiments, the present disclosure provides devices and methods for priming solid tumors with pressure pulses generated by piezophotonic materials when the materials absorb laser pulses, thereby improving the therapeutic outcome of administration of a therapeutic agent.

Prior to the present disclosure, it was totally unexpected that pressure pulses with spatial-peak temporal-average intensities below the output safety limits set by FDA for diagnostic ultrasound, which are very well tolerated by normal tissues, could change the TME. EXAMPLE 1 shows typical stress waves generated with the absorption of laser pulses in light-to-pressure (piezophotonic) transducers. EXAMPLES 2 and 3 show that such stress waves are safe for cells in vitro and for tissues in vivo. The fact that the pressure pulses of these stress waves do not produce any detectable effect in normal tissues is consistent with the fact that their spatial-peak temporal-average intensities are below the output safety limits set by FDA for diagnostic ultrasound. The pressure pulses are completely safe and do not change normal tissues. However, as presented in EXAMPLE 4, exposure of solid tumors to the pressure pulses results in solid tumor priming and enhanced response to anti-cancer therapeutic agents. The person skilled in the art could not anticipate that pressure pulses are very well tolerated by normal tissues can be very effective in solid tumor priming.

According to some embodiments, the present disclosure provides a device for tumor priming by pressure pulses comprising: a pulsed laser system; a light guide configured to direct laser pulses to one or more light-to-pressure transducers; one or more light-to-pressure transducers configured to absorb laser pulses from the pulsed laser system and generate pressure pulses; a tumor-positioning support structure configured to couple one or more light-to-pressure transducers with a selected area from a solid tumor; and a control system configured to limit the exposure of the solid tumor to the pressure pulses.

In some embodiments, the pulsed laser system has a pulse repetition rate between 0.1 Hz and 100 Hz.

In some embodiments, the device comprises a control system configured to limit the exposure of the solid tumor to the pressure pulses for a period of time from 1 second (s) to 60 minutes (min).

In some embodiments, the light guide configured to direct laser pulses to one or more light-to-pressure transducers comprises one or more optical fibers or light pipes.

In some embodiments, the light guide comprises mirrors, lenses, prisms, diffusers or polarizers, or any combination thereof.

In some embodiments, the tumor-positioning support structure is configured to couple one or more light-to-pressure transducers to an acoustic coupling element disposed between the transducers and the surface of a solid tumor.

In some embodiments, the pressure pulses are photoacoustic waves. In some embodiments, the pressure pulses are shock waves. Solid tumor priming with pressure pulses involves exposing the solid tumor to one or more pressure pulses.

Photoacoustic waves are high pressure ultrasound pulses that achieve peak compressional pressures pmax =15 MPa and frequencies extending beyond 100 MHz. The instantaneous peak intensity,

I=ρₘₐₓ²/(ρv),

where ρ is the density of the medium (water: ρ=997 kg/m³) and v is the speed of sound in the same medium (water: v=1480 m/s), of such photoacoustic waves are remarkably high: I=15 kW/cm². However, since the pulse duration of the photoacoustic wave is similar to that of the laser pulse and laser repetition rates of laser pulses with mJ energies are typically of a few hertz, the peak intensity is reached for a very small fraction of the time. For example, a laser pulse with a 10 ns duration working at 10 Hz gives a duty cycle of 10⁻⁷. This means that even when the instantaneous peak intensity is 15 kW/cm², the spatial-peak temporal-average intensity is only I_{SPTA}=1.5 mW/cm². Such photoacoustic waves are safe and below the FDA output limit for diagnostic ultrasound, 0.72W/cm² . They do not produce cavitation and their effect is mostly mechanical. It is necessary to approach 500 ns pulse durations and 100 Hz pulse repetition rates to reach the duty cycle of 5×10⁻⁵ and approach the FDA safety limit for diagnostic ultrasound.

Shock waves have in common with photoacoustic waves the fact that they carry temporary density changes inside the materials where they propagate. They differ because shock waves propagate with a velocity that is higher than the local speed of sound in the material. Herein, photoacoustic waves and shock waves are collectively designated as “pressure pulses”. The person skilled in the art knows that shock waves can be generated by a variety of process, including detonation, a projectile hitting a surface, an object travelling at supersonic speed, or an intense pulsed laser producing ablation of a target. In the context of the present disclosure, the generation of shock waves by intense laser pulses is of particular interest. The laser fluence rates (in W/m²) required to generate plasma in a given target, and consequently to generate shock waves, are higher than those required for thermoelastic expansion of the target, and consequently to generate photoacoustic waves. This means that pulsed laser generation of shock waves typically gives higher peak pressures than photoacoustic waves using the same material. Nevertheless, peak intensities of both shock waves and photoacoustic waves are reached for a very small fraction of the time. Therefore, low pulse repetition rates (≤100 Hz) lead to very low duty cycles and pressure pulses up to 100 MPa can be used without significant damage to tissues when the pressure pulses are generated by laser pulses with nanosecond duration.

According to some embodiments of the present disclosure, a superficial solid tumor can be exposed to pressure pulses by placing the material absorbing the laser pulse directly over the tumor, or over the skin layer covering the tumor, with good acoustic coupling with the skin and with the tumor. The laser pulse is directed to the material, the energy of the laser pulse is absorbed by the material and either a photoacoustic wave or a shock wave is generated on the material, crosses the material and is transmitted to the skin and to the tumor. Good acoustic coupling can be achieved with proper matching of the acoustic impedances of the material and of human tissues, and can be improved using a layer of acoustic coupling gel. The change from thermoelastic expansion generating photoacoustic waves to ablation generating shock waves depends mostly on the ablation limit of the material, on the energy of the laser pulse and on the size of the area illuminated. Reference is made to EXAMPLE 1 in the example section of the present disclosure, that describes a method to generate a photoacoustic wave and its characterization.

In many clinical situations the solid tumor is not superficial, i.e., it is more than 3 cm beneath the surface of the body. In such situations, the stress waves generated at the surface of the body may be strongly attenuated by healthy tissues before reaching the tumor mass and may lose efficiency in tumor priming. According to the FDA, the attenuation of ultrasound in tissues can be calculated with the derating factor 0.3 dB/(cm MHz). This means that 3 cm from a 3.3 MHz transducer, the derated temporal-average intensity of ultrasound is 3 dB (i.e., half of) the value measured in water. However, higher frequencies have a stronger influence in tumor priming and if the same derating factor is employed for a frequency of 33 MHz at 3 cm, the derated temporal-average intensity becomes 30 dB (i.e., 0.001 of) the value measured in water. This shows that the light-to-pressure transducers must be placed closer than 3 cm from the surface of the solid tumor to obtain a significant exposure of the solid tumor to the stress waves. The present disclosure is based, in part, on the surprising finding that normal cells and healthy tissues are not affected by stress waves. Reference is made to EXAMPLES 2 and 3 in the example section of the present disclosure, that show that normal cells and healthy tissues are not affected by stress waves.

In some embodiments, even when the solid tumor is not superficial, it may nevertheless be possible to approach the tumor mass through natural body orifices using methods of endoscopy. Minimally invasive procedures can be used to generate stress waves in the vicinity of solid tumors in the gastrointestinal tract, respiratory tract, urinary tract or female reproductive system. Stress waves can be generated close to a solid tumor in these locations because endoscopes have channels that allow for the insertion of optical fibers. Alternatively, small incisions, with a length smaller than 2 cm, can be made to give access of optical fibers to normally closed body cavities in procedures such as laparoscopy or thoracoscopy. Moreover, optical fibers can be inserted in catheters and reach many desired locations inside the human body.

According to some embodiments of the present disclosure, the use of optical fibers allows for the delivery of laser light in the vicinity of solid tumors. Non-limiting examples of solid tumors that can be reached with optical fibers include gastric cancer, enteric cancer, lung cancer, breast cancer, uterine cancer, esophageal cancer, ovarian cancer, pancreatic cancer, pharyngeal cancer, sarcomas, hepatic cancer, cancer of the urinary bladder, cancer of the upper jaw, cancer of the bile duct, head and neck cancer, cancer of the tongue, cerebral tumor, skin cancer, malignant goiter, prostatic cancer, colorectal cancer, cancer of the parotid gland, and renal cancer.

In some embodiments, when optical fibers are employed to deliver laser light to solid tumors, the laser light is directed to proximal end of the optical fiber and a piezophotonic transducer can be coupled to its distal end, which is in the vicinity of the solid tumor. The piezophotonic transducer absorbs most of the intensity of the laser pulse and generates a stress wave. A stress wave is generated each time that a laser pulse is absorbed by the piezophotonic transducer. The stress wave may be generated by thermoelastic expansion of the piezophotonic transducer, under the conditions of thermal confinement and stress confinement, and in this case the stress wave is a photoacoustic wave. The stress wave may be generated by ablation of the piezophotonic transducer, which implies the removal or destruction of some material of the transducer, and in this case the stress wave is a shock wave. In either case, the stress wave propagates in the piezophotonic transducer, from the side where the laser pulse was absorbed to the opposite side, and then is transferred to tissues in the vicinity of the solid tumor, or directly to the solid tumor.

According to the present disclosure, piezophotonic transducers can be made from a wide diversity of dyes or pigments, but to generate pressure pulses with high peak compressional amplitudes, the dyes or pigments must have high absorption coefficients (μ) at the wavelength of the laser pulse, and rapidly and efficiently transform the optical energy absorbed into thermal energy. Moreover, when the intent is to generate stress waves that are photoacoustic waves, the dyes or pigments must be preferably incorporated in a material with high Grüneisen parameter (Γ>0.5), because the peak pressure of the photoacoustic wave is given by

po=ΓμF,

where F is the local light fluence. When the intent is to generate stress waves that are shock waves, the dyes or pigments must preferably be incorporated in a material with a low ablation threshold. This is the case, for example, of poly(ethylene terephthalate), polyimide and triazene polymers. These polymers can be used to produce piezophotonic materials that undergo ablation with the production of shock waves at laser fluences below 200 mJ/cm².

Non-limiting examples of dyes or pigments that can be used to make piezophotonic materials according to the present disclosure, are ortho-hydroxybenzophenone and similar molecules undergoing ultrafast photoinduced intramolecular proton or hydrogen-atom transfers that return rapidly to the original ground state, Mn^{III} complexes of meso-tetraphenylporphyrin and other paramagnetic complexes with ultrafast metal-to-ligand and/or ligand-to-metal charge-transfer relaxation processes, complexes with charge-transfer bands that return to the ground state by ultrafast charge recombination, β-carotene and other systems that rapidly decay to the ground state through conical intersections, graphite or carbon nanoparticles or carbon nanotubes or carbon soot and other materials capable of ultrafast transfer of their electronic energy to phonon modes followed by cooling in the sub-nanosecond time scale, semiconductor materials with short-lived transient states, or other materials, or mixtures of materials, with ultrafast radiationless relaxation processes. When ablation takes place, in addition to the conversion of optical energy in thermal energy, structural volume changes of the dyes and pigments may also occur and contribute to increase the intensity of the stress waves.

In some embodiments, the light-to-pressure transducer comprises a laser light absorbing system and a material with a Grüneisen parameter higher than 0.5, and wherein each pressure pulse is a wavefront of a photoacoustic wave.

Some non-limiting examples of materials with high Grüneisen parameters (G>0.5) are polymers (polydimethylsiloxane, polystyrene, polyamide, poly(vinyl chloride), polyethylene, polyacrylonitrile, poly(ethylene terephthalate), polychloroprene, parylene), metallic films, glasses and layered materials containing them. Such materials can absorb light of a laser pulse, or be designed to incorporate dyes or pigments that absorb light of a laser pulse, in a very short optical path, which is very convenient to fabricate piezophotonic materials to work with endoscopes or catheters.

In some embodiments, the light-to-pressure transducer comprises a laser light absorbing system and a material with an ablation threshold below 200 mJ/cm², and wherein each pressure pulse is a wavefront of a shock wave.

In some embodiments, piezophotonic transducers made of dyes or pigments and of a material with a high Grüneisen parameter or a low ablation threshold can take various forms and shapes. Considering that the dyes or pigments must have high absorption coefficients, the piezophotonic transducers may absorb most of the laser pulse in an optical path shorter than 200 μm, or preferably shorter than 100 μm, or most preferably shorter than 50 μm. In view of the very small thickness of piezophotonic transducers, they can be used to cover the distal end of an optical fiber.

In some embodiments, the peak compressional pressures of the stress waves are from 0.1 MPa to 100 MPa.

In some embodiments, 90% of each pressure pulse lasts from 0.1 ns to 500 ns.

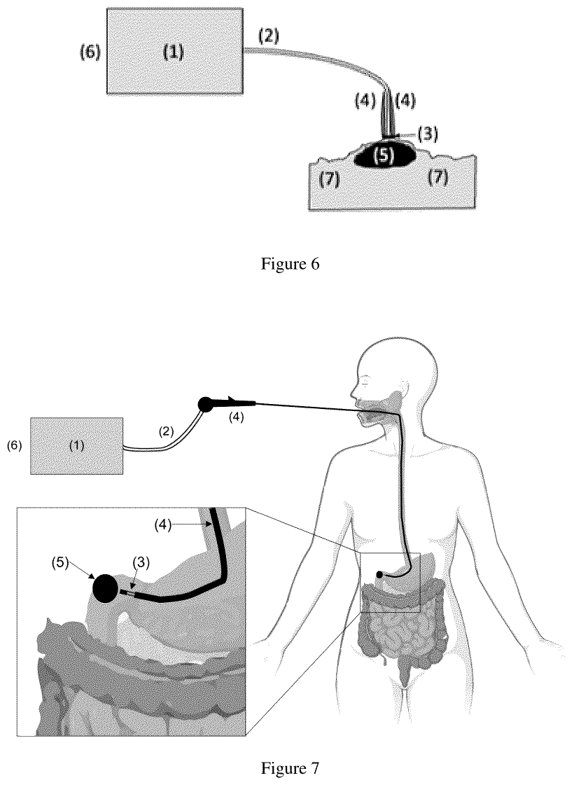

Reference is made to FIGS. 6, 7 and 8 that illustrate various embodiments of a device according to the present disclosure, wherein a piezophotonic transducer is coupled to the distal end of an optical fiber.

In some embodiments, the optical fiber is held by a tumor-positioning support structure that orients the laser pulse and a light-to-pressure transducer to a solid tumor less than 3 cm from the surface of the body (FIG. 6). In another embodiment, the optical fiber is inserted in an endoscope, that works as a tumor-positioning support structure, and at the distal end the optical fiber is optically connected to an optical diffuser and the optical diffusor is at least partially coated with a piezophotonic transducer placed within 3 cm from a solid tumor (FIG. 7). In some embodiments, the optical fiber is inserted in a catheter, that works as a tumor-positioning support structure with the assistance of a balloon, and at the distal end the optical fiber has a lens that directs the laser pulse to a piezophotonic transducer placed within 3 cm from a solid tumor (FIG. 8). In another embodiment, the optical fiber is connected to an optical diffuser, the optical diffusor is coated with a piezophotonic transducer and inserted in a solid tumor, and the system of perforating the tumor and inserting the light-to-pressure transducer is the tumor-positioning support structure and this structure has a sharp end.

In some embodiments, the tumor-positioning support structure is an endoscope and the light guide is one or more optical fibers configured to carry the laser light from the light source through the endoscope, to one or more light-to-pressure transducers at the distal ends of the optical fibers.

In some embodiments, the endoscope is configured for insertion in a hollow organ through a natural body opening or through an incision in the body with less 2 cm in length.

In some embodiments, the tumor-positioning support structure is a catheter and the light guide is one or more optical fibers configured to carry the laser light from the light source through the catheter, to one or more light-to-pressure transducers at the distal ends of the optical fibers.

In some embodiments, the catheter is configured for insertion into a body cavity, duct, vessel, brain, skin or adipose tissue.

In some embodiments, the tumor-positioning support structure comprises a sharp end configured to enable the insertion of one or more light-to-pressure transducers into the solid tumor.

Tumor priming using pressure pulses consists in bringing a piezophotonic material within 3 cm of the solid tumor, where the path between the piezophotonic material and the solid tumor is filled by a medium capable of transmitting pressure pulses, exposing the piezophotonic material to laser pulses that generate peak compressional pressures from 0.1 to 100 MPa in the piezophotonic material, and directing such pressure pulses to at least part of the solid tumor for a time in the range of 1 second to 1 hour. The laser pulses may have durations (full-width at half height) of femtoseconds, picoseconds or nanoseconds. Preferably, the laser pulse durations should be less than 500 nanoseconds because under these conditions the thermal confinement condition is more easily met and 90% of the pressure pulse lasts less 500 ns.

In some embodiments, the exposure of the solid tumors to the pressure pulses can be performed for a short period of time (e.g., 1 sec), for a long period of time (e.g., 1 hour) or for an intermediate period of time. The exposure of a solid tumor to the pressure pulses can be performed before, during or after, the administration of the therapeutic agent, and can be timed according to the plasma lifetime of the therapeutic agent.

The present disclosure is based, in part, on the finding that, in repeated administrations of the therapeutic agent, and/or for therapeutic agents with long plasma half-lives, the exposure of solid tumors to the pressure pulses can be performed several times, which can be several times a day, several times a week, several times a month, or several times a year.

The present disclosure is based, in part, on the finding that, solid tumor priming with pressure pulses as described herein, facilitates the infiltration of a therapeutic agent in a solid tumor and increases the response to therapy. Solid tumor priming by exposure to pressure pulses enhances the infiltration of a variety of therapeutic agents in a solid tumor without affecting the delivery of the therapeutic agents to healthy tissues of the host or enhancing host toxicity. It is particularly valuable for the delivery of small molecule drugs extensively bound to plasma proteins and of macromolecules, especially when they are biological pharmaceuticals. The present disclosure is based, in part, on the finding that, solid tumor priming by pressure pulses improves the therapeutic efficacy of immunotherapies, notably when the therapeutic agents are mAb employed in ICB therapy. Solid tumor priming by pressure pulses facilitates the infiltration of tumor antigen-specific T lymphocytes into tumors and their integration into the tumor microenvironment (TME), contributing to enhance tumor responses to immunotherapies. The present disclosure is based, in part, on the unexpected finding that, pressure pulses that are well tolerated by normal tissues can be effective in solid tumor priming.

According to some embodiments, the present disclosure provides a method of treating a tumor in a subject. In some embodiments, the method comprises administering to the subject (i) an amount of high-intensity photoacoustic waves and (ii) an amount of a therapeutic agent, wherein the amounts of (i) and (ii) together are sufficient to treat a solid tumor, and the order of administration can be selected from (i) before (ii), (i) simultaneously with (ii) or (i) after (ii). In some embodiments, a method of sensitizing a tumor in a subject to an amount of an anti-cancer therapy is disclosed, the method comprising of administering to the subject, prior to or during the course of the anti-cancer therapy, an amount of pressure pulses effective to improve the response of a tumor in a subject to an amount of an anti-cancer therapy administered to the subject. In some embodiments, a subject is a cancer patient.

According to some embodiments, the present disclosure provides a method for treating a solid tumor in a subject afflicted with cancer, the method comprising: pressure-pulse tumor priming of the solid tumor by exposure of the solid tumor to one or more pressure pulses, wherein the pressure pulses have peak compressional pressures from 0.1 MPa to 100 MPa, and 90% of each pressure pulse lasts from 1 ns to 500 ns; and administering of one or more anticancer therapeutic agents to the subject, thereby treating the solid tumor in a subject afflicted with cancer. In some embodiments, a subject afflicted with cancer is a cancer patient.

In some embodiments, the step of pressure-pulse tumor priming of the solid tumor by exposure of the solid tumor to one or more pressure pulses, is performed before administering of one or more anticancer therapeutic agents to the subject, during administering of one or more anticancer therapeutic agents to the subject, or after administering of one or more anticancer therapeutic agents to the subject.

In some embodiments, the method further comprises a step of repeating (i) the pressure-pulse tumor priming, (ii) the administering of one or more anticancer therapeutic agents, or both. In some embodiments, repeating is at least one time, at least 2 times, at least 3 times, at least 5 times or the number of times required to alleviate the symptoms of the subject afflicted with cancer, with doses that improve a response of the solid tumor to the treatment.

In some embodiments, the anticancer therapeutic agent is selected from the group consisting of an inhibitor to an inhibitory checkpoint molecule, an activator of a stimulatory checkpoint molecule, an antibody, a cytokine, an interferon, an interleukin, a vaccine, an oncolytic virus, chimeric antigen receptor T cells, and any combination thereof.

In some embodiments, the therapeutic agent is a biological therapeutic.

In some embodiments, the therapeutic agent is a monoclonal antibody (mAb) used to treat cancer, or any combination of mAbs used to treat cancer.

In some embodiments, the therapeutic agent is selected from ipilimumab, pembrolizumab, nivolumab, atezolizumab, durvalumab, avelumab, cemiplimab, dostarlimab, tislelizumab, relatlimab, toripalimab, camrelizumab, sintilimab, or any combination thereof.

In some embodiments, the therapeutic agent is a cytostatic or a cytotoxic drug bound to a plasma protein.

In some embodiments, the therapeutic agent is a macromolecule.

In some embodiments, the therapeutic agent is a nanomedicine.

According to some embodiments, the present disclosure provides a kit comprising a device as described hereinabove, and an anticancer therapeutic agent as described hereinabove.

According to some embodiments, the present disclosure provides a system comprising at least one device as described hereinabove.

According to some embodiments, the present disclosure provides a device comprising means for generating pressure pulses having peak compressional pressures from 0.1 MPa to 100 MPa, and 90% of each pressure pulse lasts from 1 ns to 500 ns, for treating a solid tumor in a cancer patient. In some embodiments, the device is the device as described hereinabove.

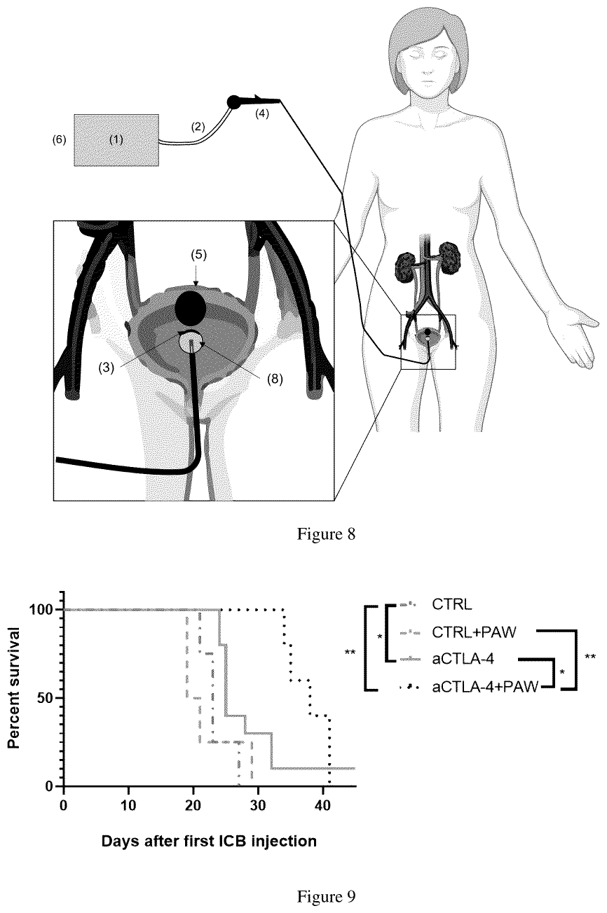

Pressure-pulse tumor-priming therapy (PPTPT) combines the exposure of solid tumors to pressure pulses, as described hereinabove, with the administration of a therapeutic agent with an anti-cancer effect. Reference is made to EXAMPLE 4 and FIG. 9 that illustrate PPTPT of mice with orthotopic 4T1 mammary carcinomas. 4T1 cells are inoculated in the mammary fat pad of BALB/c mice and allowed to grow to 3 mm in the longest diameter before the beginning of the interventions. Pressure-pulse tumor priming in this example consisted in the exposure of the orthotopic tumor to photoacoustic waves for 5 minutes on days 0 and 2. Therapy in this example consisted in the intraperitoneal administration of anti-CTLA- 4 mAbs (aCTLA 4) on days 0, 2, 6 and 10. Control groups show that the survival of mice in the group subject to tumor priming only (priming, no treatment) are not statistically different from those of the control group (no priming, no treatment). In the group subject to the four administrations of aCTLA4 (no priming, treatment) only one animal responded to therapy. On the contrary, all the animals in the group subject to PPTPT (priming, therapy) responded to the therapy.

The extraordinary achievement of tumor priming with photoacoustic waves can be realized comparing PPTPT with the tumor priming using angiotensin receptor blockers [17]. The median survival time of mice bearing orthotopic 4T 1 tumors increases from 20 days in the combination of aCTLA 4 plus aPD 1, to 24 days when this combination is associated with tumor priming with angiotensin receptor blockers [17]. In the same animal model, i.e., mice bearing orthotopic 4T1 tumors, EXAMPLE 4 shows that the median survival time increases from 21 days when the animals are treated with aCTLA4 to 36 days when photoacoustic priming is associated with the aCTLA4 treatment. The person skilled in the art could not anticipate that exposing orthotopic 4T1 tumors to photoacoustic waves for 5 minutes in two independent sessions could increase by 15 days the median survival time of mice bearing orthotopic 4T1 tumors, especially considering that priming by angiotensin receptor blockers increases the median survival time by 4 days only. Orthotopic 4T1 tumors are widely recognized as very difficult to treat and increasing the response to immunotherapy in all the mice with just two sessions of 5 minutes local exposure of the tumors to harmless photoacoustic waves is totally unexpected.

The present disclosure is based, in part, on the finding that PPTPT is a novel and surprising effective approach to increase solid tumor response to therapeutic agents. High-intensity broadband stress waves exert mechanical forces at the microscopic level that can remodel the TME. As illustrated in EXAMPLE 1, peak pressures of ~7 MPa of pressure waves with relevant frequencies of about 20 MHz, correspond to changes of 50 bar in 10 nsec or, considering the speed of sound propagation in tissues, a change of 50 bar in 15 μm. Dramatic pressure changes are produced on the scale of the size of a cell. The present disclosure is based, in part, on the finding that cells survive these high pressures, as shown in EXAMPLE 2, and normal tissues do not exhibit any adverse effects, as shown in EXAMPLE 3. However, the mechanical forces exerted by such pressure pulses in the TME enable micro-mechanic priming of solid tumors, as shown in EXAMPLE 4.

Various embodiments and aspects of the present disclosure as delineated hereinabove and as claimed in the claims section below find experimental support in the following examples.

### REFERENCES CITED

[1] I. Mellman, G. Coukos, G. Dranoff, Cancer immunotherapy comes of age, Nature, 480 (2011) 480-489.
[2] J. M. Kirkwood, L. H. Butterfield, A. A. Tarhini, H. Zarour, P. Kalinski, S. Ferrone, Immunotherapy of Cancer in 2012, CA Cancer J. Clin., 62 (2012) 309-335.
[3] S. J. Casak, L. Marcus, L. Fashoyin-Aje, S. Mushti, J. Cheng, Y.-L. Shen, W. F. Pierce, L. Her, K. B. Goldberg, M. R. Theoret, P. G. Kluetz, R. Pazdur, S. J. Lemery, FDA Approval Summary: Pembrolizumab for the First-line Treatment of Patients with MSI-H/dMMR Advanced Unresectable or Metastatic Colorectal Carcinoma, Clin. Cancer Res., 27 (2021) 4680-4684.
[4] J. D. Twomey, B. Zhang, Cancer Immunotherapy Update: FDA-Approved Checkpoint Inhibitors and Companion Diagnostics, AAPS J., 23 (2021) 39.
[5] A. Haslam, V. Prasad, Estimation of the Percentage of US Patients With Cancer Who Are Eligible for and Respond to Checkpoint Inhibitor Immunotherapy Drugs, JAMA Network Open, 2 (2019) e192535.
[6] J. M. Fritz, M. J. Lenardo, Development of immune checkpoint therapy for cancer, J. Exp. Med., 216 (2019) 1244-1254.
[7] A. D. Waldman, J. M. Fritz, M. J. Lenardo, A guide to cancer immunotherapy: from T cell basic science to clinical practice, Nat. Rev. Immunol., 20 (2020) 651-668.
[8] A. I. Minchinton, I. F. Tannock, Drug penetration in solid tumours, Nat. Rev. Cancer, 6 (2006) 583-592.
[9] J. T. Ryman, B. Meibohm, Parmacokinetics of monoclonal antibodies, CPT Pharmacometrics Syst. Pharmacol., 6 (2017) 576-588.
[10] M. Centanni, D. J. A. R. Moes, I. F. Troconiz, J. Ciccolini, J. G. C. van Hasselt, Clinical Pharmacokinetics and Pharmacodynamics of Immune Checkpoint Inhibitors, Clin. Pharmacokinet., 58 (2019) 835-857.
[11] Y. Matsumura, H. Maeda, A New Concept for Macromolecular Therapeutics in Cancer Chemotherapy: Mechanism of Tumoritropic Accumulation of Proteins and the Antitumor Agent Smancs, Cancer Res., 46 (1986) 6387-6392.
[12] Y. H. Bae, K. Park, Targeted drug delivery to tumors: Myths, reality and possibility, J. Control. Release, 153 (2011) 198-295.
[13] Y. Huang, J. Yuan, E. Righi, W. S. Kamoun, M. Ancukiewicz, J. Nezivar, M. Santosuosso, J. D. Martin, M. R. Martin, F. Vianello, P. Leblanc, L. L. Munn, P. Huang, D. G. Duda, D. Fukumura, R. K. Jain, M. C. Poznansky, Vascular normalizing doses of antiangiogenic treatment reprogram the immunosuppressive tumor microenvironment and enhance immunotherapy, Proc. Natl. Acad. Sci. USA, 109 (2012) 17561-17566.
[14] I. A. Khawar, J. H. Kim, H.-J. Kuh, Improving drug delivery to solid tumors: Priming the tumor microenvironment, J. Control. Release, 201 (2015) 78-89.
[15] T. Stylianopoulos, J. D. Martin, V. P. Chauhan, S. R. Jain, B. Diop-Frimpong, N. Bardeesy, B. L. Smith, C. R. Ferrone, F. J. Hornicek, Y. Boucher, L. L. Munn, R. K. Jain, Causes, consequences, and remedies for growth-induced solid stress in murine and human tumors, Proc. Natl. Acad. Sci. USA, 109 (2012) 15101-15108.
[16] H. Raskov, A. Orhan, J. P. Christensen, I. Gögenur, Cytotoxic CD8⁺T cells in cancer immunotherapy, Br. J. Cancer, 124 (2021) 359-367.
[17] V. P. Chauhan, Y. X. Ivy X. Chen, R. Tong, M. R. Ng, J. D. Martin, K. Naxerova, M. W. Wu, P. Huang, Y. Boucher, D. S. Kohane, R. Langer, R. K. Jain, Reprogramming the microenvironment with tumor-selective angiotensin blockers enhances cancer immunotherapy, Proc. Natl. Acad. Sci. USA, 116 (2019) 10674-10680.
[18] H.-C. Huang, I. Rizvi, J. Liu, S. Anbil, A. Kalra, H. Lee, Y. Baglo, N. Paz, D. Hayden, S. Pereira, B. W. Pogue, J. Fitzgerald, T. Hasan, Photodynamic Priming Mitigates Chemotherapeutic Selection Pressures and Improves Drug Delivery, Cancer Res., 78 (2018) 558-571.
[19] C. Guha, S. Barry, F. Macian, Low intensity focused ultrasound for treating cancer and metastasis, in: U.S. Patent (Ed.), Montefiore Medical Center, USA, 2021; U.S. Pat. No. 10,974,077.

### EXAMPLES

Reference is now made to the following examples, which together with the above descriptions illustrate some embodiments of the disclosure in a non-limiting fashion.

### Example 1

### Generation of Photoacoustic Waves with 10 MPa Peak Pressures

Carbon nanoparticles are very convenient light-absorbing systems because they strongly absorb light over a large range of ultraviolet-visible-infrared wavelengths. Carbon nanoparticles are difficult to disperse in solution, hence 160 mg of the carbon nanoparticles, produced as candle soot, were added to 5 mL of toluene and sonicated, using a tip sonicator, for 5 minutes at 60 MHz. Immediately after mechanical sonication, 2 mg of polystyrene were added to the suspension and heated in a water bath to 60°C. Polystyrene has a high Grüneisen parameter (G~0.7) and is very convenient to make thin piezophotonic transducers. Polystyrene films with dispersed carbon nanoparticles were fabricated using a mechanical applicator (Elcometer) and dried overnight to allow the remaining solvent evaporate.

Alternatively, piezophotonic transducer were produced depositing carbon nanoparticles from the combustion of a paraffin lamp on a borosilicate glass window. This glass window was directly exposed to the flame for 2 min to collect carbon soot. Then, the thin layer of carbon soot deposited on the window was covered with 0.1 mL of polydimethylsiloxane (PDMS) and subject to vacuum for 10 min to remove any air bubbles. Next, a weight of 100 g was placed over the system (glass+soot+PDMS) to make a thin layer of PDMS and exposed for 10 additional minutes to vacuum to remove the excess of air trapped in the system. Finally, the complete assembly system was heated in an oven at 50 °C overnight to obtain a full cure of PDMS.

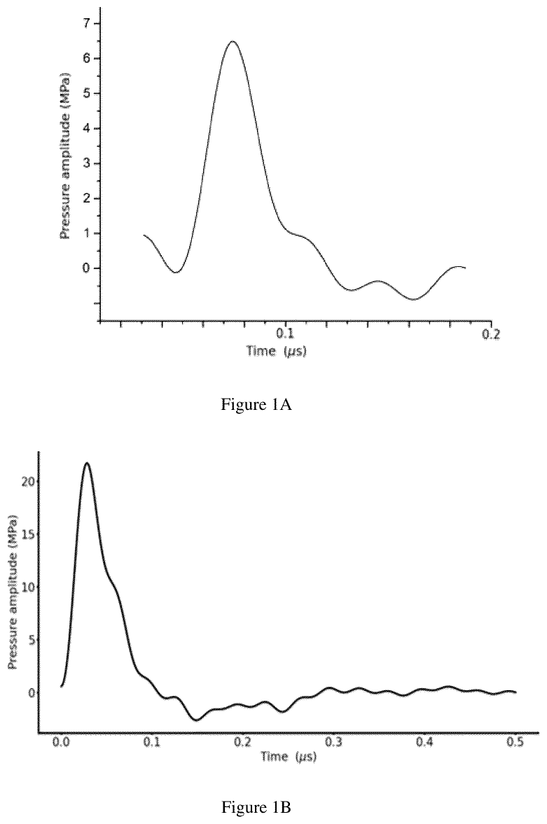

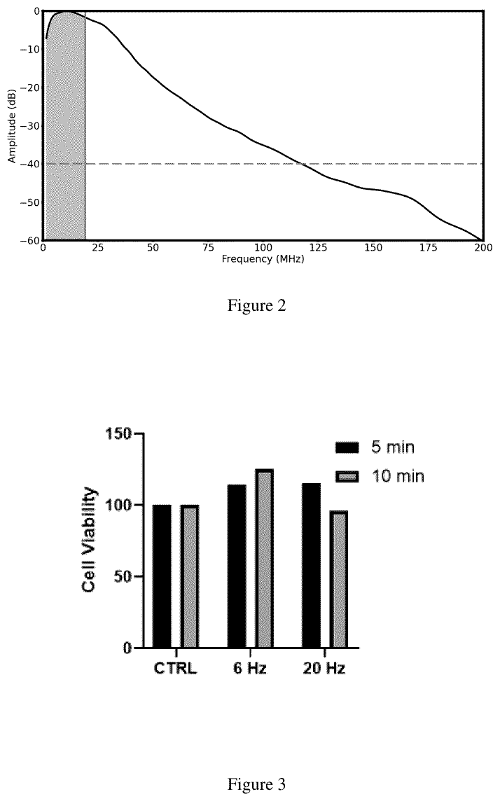

Piezophotonic transducers made with carbon nanoparticles and polystyrene or PDMS as described above, were investigated with pulsed laser excitation at 1064 nm to characterize the pressure pulses they can generate. Laser excitation employed a Nd: YAG laser (Monfort M-NANO) with nanoseconds pulses to generate photoacoustic waves. Two types of ultrasound measurements were made. Absolute pressures were measured using a 0.2 mm needle hydrophone (Precision Acoustics, model NH0200), calibrated to the 1 to 30 MHz range. Ultrasonic frequency distributions were investigated with a 225 MHz contact transducer (Panametrics/Olympus, model V2113). FIGS. 1A-B show the absolute pressure pulses obtained with laser fluences of ~60 mJ/cm² and ~250 mJ/cm², measured with the hydrophone. FIG. 2 shows the ultrasonic frequency distribution using a laser fluence of ~60 mJ/cm² measured with the contact transducer.

### Example 2

### Photoacoustic Waves with Peak Pressures of 10 MPa Are Not Toxic to Fibroblasts in Vitro

Monolayers of immortalized monkey fibroblast cell line (COS-7) were cultured in Dulbecco's Modified Eagle's Medium (DMEM) supplemented with 10% heat-inactivated fetal bovine serum (Gibco) and 1% penicillin and streptomycin (Invitrogen), in humidified atmosphere with 5% CO₂ at 37 °C. COS- 7 cells were first seeded in 12-weel plates at a density of 30.000 cells/well in 2 mL of medium; 24 h later, the medium was changed to enable rapid growth of the cells; 48 h after seeding, the medium was removed and 300 μL of new culture medium was added. Next, the piezophotonic material of EXAMPLE 1 was immersed in the culture medium and placed within 3 mm from the surface of the COS-7 cells monolayer. Then, the cells were exposed to photoacoustic waves for 5 minutes (5 mins) or for 10 minutes (10 mins), at the laser repetition rates of 6 Hz or 20 Hz and laser fluence ~60mJ/cm² , using a Nd: YAG laser (Monfort M-NANO). Cell viability was measured 24 h after exposure to photoacoustic waves using the Alamar Blue® assay.

### Example 3

### Exposure of Healthy Tissues of Rats to Photoacoustic Waves 5 Min a Day, 5 Days a Week for 4 Weeks, Does Not Elicit Adverse Effects

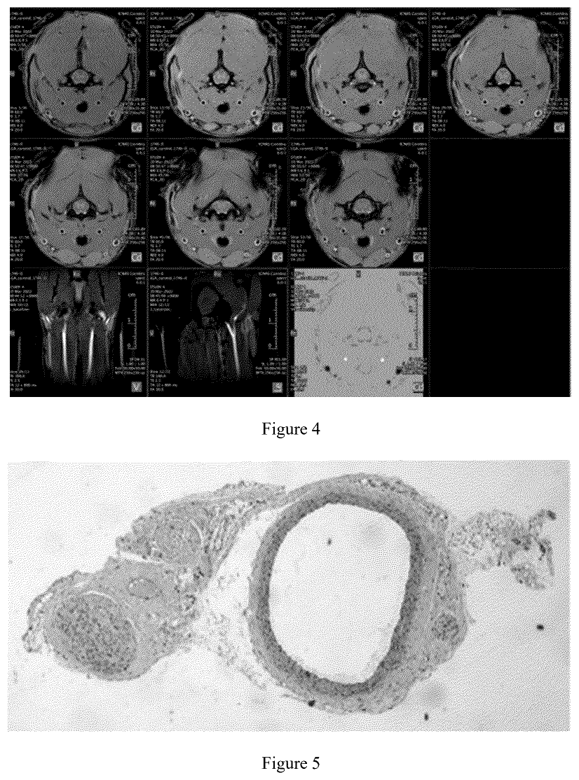

The Portuguese Animal Health Authority approved the animal experiments (DGAV authorization 0420/000/000/2011). This study employed male Sprague Dawley rats (Charles River Laboratories, Barcelona, Spain). The rats were depilated around the neck and a circle was drawn in the area to be subject to stress waves. The exposure to stress waves was performed 5 days a week, for 4 weeks. In each exposure, stress waves were generated for 5 min at 20 Hz with piezophotonic transducers made of carbon nanoparticles and PDMS and using a Monfort M-NANO Nd: YAG laser. Under the conditions employed, the peak compressional pressure of each pulse was ~3 MPa. Acoustic coupling between the piezophotonic transducer and the neck of the rat was optimized using ultrasound gel (Eco Supergel). The carotids were imaged with Magnetic Resonance Imaging (FIG. 4). Histology of sections of the neck was made at the end of the experiment (FIG. 5).

### Example4

### Pressure-Pulse Tumor-Priming Therapy

The Portuguese Animal Health Authority approved the animal experiments (DGAV authorization 0420/000/000/2011). 4T 1 cells (ATCC CRL- 2539) were cultured in Dulbecco's Modified Eagle's medium (DMEM) (Sigma-Aldrich, Saint-Louis, MO, USA) supplemented with 10% (v/v) heat-inactivated fetal bovine serum (GIBCO™, Life Technologies, Bleiswijk, The Netherlands), 100 U/mL penicillin, and 100 ng/ml streptomycin (Invitrogen™, Thermo Fisher Scientific, Grand Island, NY, USA). Tumors were established by orthotopic injection of 20,000 4T1 cells in the right mammary gland of female BALB/c mice ca. 8 to 12 weeks old (20 g).

Prior to tumor priming, the mice were depilated in the abdominal area, namely the mammary gland where the tumor was inoculated. The piezophotonic transducer employed was prepared with carbon nanoparticles and polydimethylsiloxane. The piezophotonic transducer was positioned over the tumor, and acoustic coupling was improved with a layer of acoustic coupling gel between the tumor and the piezophotonic transducer. Photoacoustic waves were generated directing laser pulses from the Monfort M-NANO Nd: YAG laser, with a laser repetition rate of 20 Hz, to the piezophotonic transducer. Under the conditions employed, the peak compressional pressure of each pulse was ~6.5 MPa.

Four study groups with 4 or 5 animals each were used in this protocol: (i) control group with orthotopic tumor, no tumor priming and no treatment; (ii) priming control group with orthotopic tumor, tumor priming with pressure pulses and no treatment; (iii) anti-mouse CTLA4 treatment group with orthotopic tumor, no tumor priming and treatment with InVivo mAb anti-mouse CTLA4 (CD152); (iv) tumor-priming treatment group with orthotopic tumor, tumor priming with pressure pulses and treatment with In Vivo mAb anti-mouse CTLA 4 (CD152). Day 0 (zero) was defined as the day of the first treatment and the tumors in all the groups had ~3 mm diameter. Day 0 corresponds to 8 days after the inoculation of the orthotopic tumor.

Group (i) was not subject to tumor priming or treatment and the orthotopic tumors grew naturally. Group (ii) was subject to tumor priming by exposing the tumors to photoacoustic waves for 5 min in days 0 and 2. Group (iii) was treated with InVivo mAb anti-mouse CTLA4 (CD152) on days 0, 2, 6 and 10, by intraperitoneal injection of In VivoMab anti-mouse CTLA4 (CD152) (Bio Cell, Lebanon, NH, USA). Group (iv) was subject to the same treatment protocol as group (iii) but additionally tumor priming was performed 10 minutes after antibody administration on days 0 and 2, as done in group (ii). The tumors were measured twice a week with a caliper and when tumors reached a diameter of 12 mm, the animals were euthanized. FIG. 9 shows the survival of mice until this endpoint.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A device for tumor priming by pressure pulses comprising:
a pulsed laser system with a pulse repetition rate from 0.1 Hz to 100 Hz;
a light guide configured to direct laser pulses to one or more light-to-pressure transducers;
one or more light-to-pressure transducers configured to absorb laser pulses from said pulsed laser system and generate pressure pulses, wherein said pressure pulses have peak compressional pressures from 0.1 MPa to 100 MPa, and 90% of each pressure pulse lasts from 0.1 ns to 500 ns;
a tumor-positioning support structure, configured to couple one or more light-to-pressure transducers with a selected area from a solid tumor, at a distance shorter than 3 cm from said area; and
a control system configured to limit an exposure of said solid tumor to said pressure pulses for a period of time from 1 second to 60 minutes.

2. The device according to claim 1, wherein said light guide comprises one or more optical fibers or light pipes, mirrors, lenses, prisms, diffusers, polarizers, or any combination thereof.

3. (canceled)

4. The device according to claim 1, wherein said light-to-pressure transducer comprises a laser light absorbing system and a material with a Grüneisen parameter higher than 0.5, and wherein each pressure pulse is a wavefront of a photoacoustic wave, or said light-to-pressure transducer comprises a laser light absorbing system and a material with an ablation threshold below 200 mJ/cm², and wherein each pressure pulse is a wavefront of a shock wave.

5. (canceled)

6. The device according to claim 1, wherein said tumor-positioning support structure is configured to hold one or more light-to-pressure transducers together with an acoustic coupling element disposed between said transducers and a surface of a solid tumor;
or said tumor-positioning support structure is an endoscope and the light guide is one or more optical fibers configured to carry a laser light from a light source through the endoscope, to one or more light-to-pressure transducers at distal ends of said optical fibers; or said tumor-positioning support structure is a catheter and the light guide is one or more optical fibers configured to carry a laser light from a light source through the catheter, to one or more light-to-pressure transducers at distal ends of the optical fibers, preferably wherein the catheter is configured for insertion into a body cavity, duct, vessel, brain, skin or adipose tissue; or wherein said tumor-positioning support structure comprises a sharp end configured to enable an insertion of one or more light-to-pressure transducers into said solid tumor.

7. (canceled)

8. The device according to claim 67, wherein said tumor-positioning support structure is an endoscope and said endoscope is configured for insertion in a hollow organ through a natural body opening or through an incision in the body with less 2 cm in length.

9. (canceled)

10. (canceled)

11. (canceled)

12. A method for treating a solid tumor in a subject afflicted with cancer, the method comprising:
pressure-pulse tumor priming of the solid tumor by exposure of said solid tumor to one or more pressure pulses, wherein said pressure pulses have peak compressional pressures from 0.1 MPa to 100 MPa, and 90% of each pressure pulse lasts from 1 ns to 500 ns; and
administering one or more anticancer therapeutic agent to said subject, thereby treating said solid tumor in a subject afflicted with cancer;
optionally repeating said pressure-pulse tumor priming, said administering of one or more anticancer therapeutic agents, or both, at least one time with doses that improve a response of said solid tumor to the treatment.

13. The method according to claim 12, wherein said pressure-pulse tumor priming of the solid tumor is performed with the device according to claim 1.

14. (canceled)

15. The method according to claim 12, wherein said anticancer therapeutic agent is selected from the group consisting of an inhibitor to an inhibitory checkpoint molecule, an activator of a stimulatory checkpoint molecule, an antibody, a cytokine, an interferon, an interleukin, a vaccine, an oncolytic virus, chimeric antigen receptor T cells, and any combination thereof or wherein said therapeutic agent is a biological therapeutic or wherein said therapeutic agent is a monoclonal antibody (mAb) used to treat cancer, or any combination of mAbs used to treat cancer or wherein said therapeutic agent is selected from ipilimumab, pembrolizumab, nivolumab, atezolizumab, durvalumab, avelumab, cemiplimab, dostarlimab, tislelizumab, relatlimab, toripalimab, camrelizumab, sintilimab, or any combination thereof or wherein said therapeutic agent is a cytostatic or a cytotoxic drug bound to a plasma protein or wherein said therapeutic agent is a macromolecule or wherein said therapeutic agent is a nanomedicine.

16. (canceled)

17. (canceled)

18. (canceled)

19. (canceled)

20. (canceled)

21. (canceled)

22. A device comprising means for generating pressure pulses having peak compressional pressures from 0.1 MPa to 100 MPa, and 90% of each pressure pulse lasts from 1 ns to 500 ns, for treating a solid tumor in a subject afflicted with cancer.

23. The device according to claim 22, wherein said device is the device according to claim 1.

24. (canceled)

25. A kit comprising a device according to any one of claims 1 to 11, and an anticancer therapeutic agent, wherein said anticancer therapeutic agent is selected from the group consisting of an inhibitor to an inhibitory checkpoint molecule, an activator of a stimulatory checkpoint molecule, an antibody, a cytokine, an interferon, an interleukin, a vaccine, an oncolytic virus, chimeric antigen receptor T cells, and any combination thereof or wherein said therapeutic agent is a biological therapeutic or wherein said therapeutic agent is a monoclonal antibody (mAb) used to treat cancer, or any combination of mAbs used to treat cancer or wherein said therapeutic agent is selected from ipilimumab, pembrolizumab, nivolumab, atezolizumab, durvalumab, avelumab, cemiplimab, dostarlimab, tislelizumab, relatlimab, toripalimab, camrelizumab, sintilimab, or any combination thereof or wherein said therapeutic agent is a cytostatic or a cytotoxic drug bound to a plasma protein or wherein said therapeutic agent is a macromolecule or wherein said therapeutic agent is a nanomedicine.

26. (canceled)

27. (canceled)

28. (canceled)

29. (canceled)

30. (canceled)

31. (canceled)

32. (canceled)
